(19) United States
(12) Patent Application Publication
(10) Pub. No.: US 2011/0034877 A1
(43) Pub. Date: Feb. 10, 2011

(54) Pouch for Containing Medical Connector Fittings

(76) Inventor: Michelle A. Salerno, Lombard, IL (US)

Correspondence Address: Bishop & Diehl, Ltd., 1750 East Golf Road, Suite 390, Schaumburg, IL, 60173, US

(21) Appl. No.: 12/537,405

(22) Filed: Aug. 7, 2009

(51) Int. Cl. A45F 3/00; A61M 39/10

(52) U.S. Cl. 604/174; 224/600

(57) ABSTRACT

An apparatus for containing a medical connector fitting used, in administering a medication about the neck of a patient. In an embodiment, a pouch is constructed of a soft compliant material and includes a compartment for storing the medical connector fitting. Furthermore, a pair of straps extend from the pouch and extend around the neck of the patient.

### TECHNICAL FIELD OF THE INVENTION

The present invention relates to medical devices, and more specifically to a pouch that allows for ready or immediate access to a medical connector fitting or coupler that is attached, via medical tubing, to a patient, while also providing for comfortable storage of the connector fitting when not in use

### BACKGROUND OF THE INVENTION

A connector fitting or coupler for an intravenous catheter can be used in administering a medication to a patient or withdrawing a fluid sample such a blood. In particular, the connector fitting can be attached, or be a part of, the catheter which includes tubing attached by a needle or the like to a vein of the patient. Typically, when not in use, the connector fitting is attached, to the patient by taping it to the patient's skin patient so that the needle or catheter does not need to be reinserted into the patient's vein. However, over time, the tape can become uncomfortable and cause skin irritation. Furthermore, removal of the tape from the patient's skin can be hurtful.

In some medical procedures, the intravenous catheter can be attached to the patient for an extended period of time. For instance, in a Hickman procedure, art intravenous catheter, also referred to as a Hickman line, can be attached to the patient for months or even years to provide for long-term intravenous access. As such, the line and connector fitting suspended from the patient can be painful, especially when inadvertently pulled upon or tugged.

### SUMMARY OF THE INVENTION

An apparatus is provided for allowing ready or immediate access to a medical connector fitting that is coupled, via medical tubing, to a patient, while also providing for comfortable storage of the connector fitting when it is not in use. In an embodiment, a pouch is constructed of a soft compliant material and includes a compartment for storing the medical connector fitting. Furthermore, a pair of straps extend from the pouch and extend around the neck of the patient.

In another embodiment, an apparatus is provided comprising a pouch having a compartment wherein a medical connector fitting can be stored therein. Further, a pair of straps extend from the pouch and around the neck of the patient wherein the distal ends of the straps are attached together.

In yet another embodiment, an apparatus is provided comprising a pouch having a compartment with an opening thereto. In addition, intravenous catheter tubing passes through the opening wherein the intravenous catheter tubing connector fitting is contained within the compartment. Moreover, a pair of straps extend from the pouch with the ends of the straps being attached, together.

Other embodiments, systems, methods, features, and advantages of the present invention will be, or will become, apparent to one having ordinary skill in the art upon examination of the following drawings and detailed, description. It is intended that all such additional systems, method, features, and advantages be within the scope of the present invention, and can be protected by the accompanying claims.

### BRIEF DESCRIPTION OF THE DRAWINGS

The invention may be better understood with reference to the following drawings. The components in the drawings are not necessarily to scale, emphasis instead being placed upon illustrating the principles of the invention. In the drawings, like reference numbers designate corresponding parts throughout.

FIG. 1 is an elevation view of a pouch in accordance with the present invention worn about the neck of a patient and containing a medical connector fitting therein; and,

FIG. 2 is a plan view of the pouch of FIG. 1 with the medical connector fitting stored therein.

### DETAILED DESCRIPTION OF THE INVENTION

The following descriptions of detailed embodiments are for exemplifying the principles and advantages of the inventions. They are not to be taken in any way as limitations on the scope of the inventions.

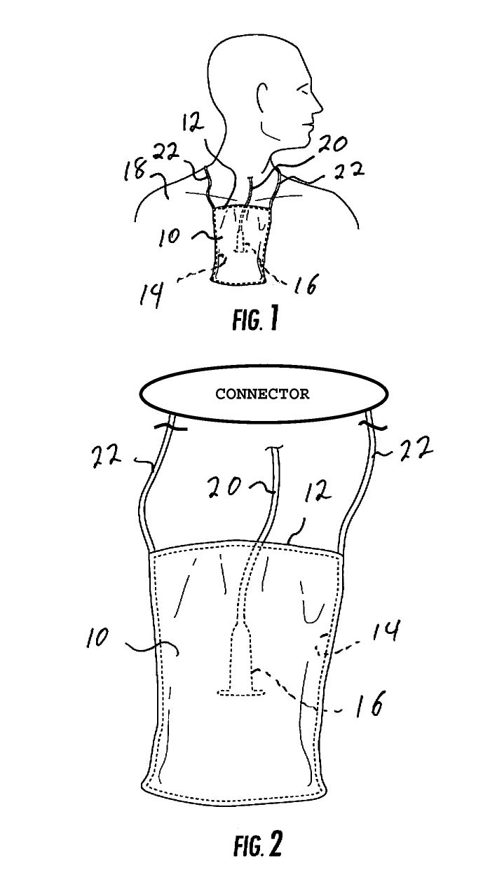

In one application of an embodiment in accordance with the present invention, an apparatus comprising a pouch is provided for storing a medical connector fitting when it is not in use. Turning to FIGS. 1 and 2, an elevation view and plan view, respectively, are provided of the pouch in accordance with the present invention wherein the pouch is worn about the neck of a patient.

The pouch 10 is preferably constructed of a soft compliant material such as cotton or another type of fabric that does not cause skin irritation. The pouch 10 is generally rectangular with an opening or mouth 12 provided, at only one end (e.g., the top) of the pouch. Accordingly, the pouch 10 provides a cavity or compartment 14 for receiving a medical connector fitting 16.

The medical connector fitting 16 can be a conventional fitting for administering a medication to a patient 18. In an embodiment, the connector 16 can be a conventional leur fitting or the like that is coupled to medical tubing 20. In turn, the medical tubing 20 is connected, in a conventional manner, to a vein of the patent.

Attached to the pouch 10 are two straps 22 that are about sixteen inches in length, each. The straps 22 can be constructed of the same material as the pouch, or a different material It is preferred, however, that the straps be constructed of a soft compliant material such as cotton or another type of fabric that does not cause skin irritation. Moreover, in an embodiment, the straps 22 can be elastic.

The proximal ends of the straps 22 can be attached to each side of the mouth 12 of the pouch 10. Accordingly, the straps 22 can be sewn or integrally attached to the pouch 10. In an embodiment, the distal ends 24 of the straps 22 are attached together behind the neck 24 of the patient 18 to thus secure the pouch about the patient's neck.

The straps 22 can be attached together by tying them to each other. Alternatively, the distal ends of the straps 22 can be attached to a fastener or clasp (not shown) for securing the ends of the straps together whereby they extend around the neck of the patient.

As previously indicated above, the straps 22 of the pouch 10 can be connected together about the neck of the patient 18. The medical connector fitting 16 can then be stored, in the compartment 14 of the pouch. When the medical connector fitting 16 is needed, in a procedure to apply medication to the patient or withdraw fluid, the medical connector fitting 16 can be removed from the pouch compartment. Furthermore, if desired, the pouch can be removed from about the neck of the patient 18 by disconnecting the ends of the straps 22 from each other.

It should be emphasized that the above-described embodiments of the present invention, particularly, any “preferred.” embodiments, are possible examples of implementations merely set forth for a clear understanding of the principles for the invention. Accordingly, many variations and modifications may be made to the above-described embodiments) of the invention without substantially departing from the spirit and principles of the invention. All such modifications are intended to be included herein within the scope of this disclosure and the present invention, and protected by the following claims.

1. An apparatus for containing a medical connector fitting used in administering a medication about the neck of a patient comprising:
a pouch constructed of a soft compliant material and having a compartment with an opening thereto wherein the connector fitting is contained within the compartment;
a pair of straps having distal ends and constructed of a soft compliant material extending from the pouch wherein the distal ends are attached together and extend around the neck of the patient; and,
wherein the opening to the pouch is between the straps and tubing extends from about the neck of the patient, through the opening, and is attached to the connector fitting.

2. The apparatus of claim 1 wherein the pouch and the straps are constructed of the same soft compliant material.

3. The apparatus of claim 1 wherein the medical connector fitting is a luer fitting.

4. The apparatus of claim 1 wherein the medical connector fitting is not a luer fitting.

5. The apparatus of claim 1 wherein the straps are elastic.

6. The apparatus of claim 1 wherein the distal ends of the straps are tied together.

7. The apparatus of claim 1 wherein a connector is coupled to the distal ends of the straps.

8. An apparatus for storing a medical connector fitting used in administering a medication about the neck of a patient comprising:
a pouch having a compartment with an opening and the medical connector fitting contained therein;
a pair of straps extending from the pouch and around the neck of the patient;
wherein the straps have distal ends that are attached together; and,
wherein the opening to the pouch is between the straps and tubing extends from about the neck of the patient, through the opening, and is attached to the connector fitting.

9. The apparatus of claim 8 wherein the pouch and the straps are constructed of a soft compliant material.

10. The apparatus of claim 8 wherein the medical connector fitting is a luer fitting.

11. The apparatus of claim 8 wherein the medical connector is not a luer fitting.

12. The apparatus of claim 8 wherein the straps are elastic.

13. The apparatus of claim 8 wherein the distal ends of the straps are tied together.

14. The apparatus of claim 8 wherein a connector is coupled to the distal ends of the straps.

15. An apparatus for keeping a connector fitting attached to IV tubing comprising:
a pouch having a compartment with an opening thereto wherein the IV tubing passes through the opening and the connector fitting is contained within the compartment;
a pair of straps extending from the pouch wherein the distal ends are attached together; and
wherein the opening to the pouch is between the straps.

16. The apparatus of claim 15 wherein the pouch and the straps are constructed of a soft compliant material.

17. The apparatus of claim 15 wherein the connector fitting is a luer fitting.

18. The apparatus of claim 15 wherein the connector fitting is not a luer fitting.

19. The apparatus of claim 15 wherein the straps are elastic.

20. The apparatus of claim 15 wherein a connector is coupled to the distal ends of the straps.
